(19) United States
(12) Patent Application Publication
(10) Pub. No.: US 2020/0069702 A1
(43) Pub. Date: Mar. 5, 2020

(54) ORAL PHARMACEUTICAL COMPOSITIONS COMPRISING AN UNMICRONIZED SELECTIVE PROGESTERONE RECEPTOR AS ACTIVE AGENT

(71) Applicant: Repros Therapeutics Inc., Medison, NJ (US)

(72) Inventor: Joseph S. Podolski, The Woodlands, TX (US)

(21) Appl. No.: 16/468,256

(22) PCT Filed: Dec. 7, 2017
(86) PCT No.: PCT/US2017/065108

§ 371 Date: Jun. 10, 2019

Related U.S. Application Data
(60) Provisional application No. 62/432,069, filed on Dec. 9, 2016.

(51) Int. Cl. A61K 31/573; A61K 9/48; A61P 15/00

(52) U.S. Cl. CPC A61K 31/573; A61P 15/00; A61K 9/4866; A61K 9/4825

(57) ABSTRACT

The subject matter of the present invention is pertinent to the field of oral delivery of pharmaceutically active agents. Embodiments of the instant invention disclose a liquid or semi-solid non-aqueous oral pharmaceutical fill composition comprising (a) a pharmaceutically effective amount of an unmicronized selective progesterone receptor modulator (SPRM) and (b) a polyethylene glycol (PEG) as well as methods for treating a variety of progesterone related disorders by orally administering such a composition.

### FIELD OF THE INVENTION

In several embodiments, the present invention relates to oral pharmaceutical compositions comprising a selective progesterone receptor modulator and their use for the treatment of a variety of progesterone related conditions.

### BACKGROUND OF THE INVENTION

The effect of the steroid hormone progesterone on the reproductive system has been well-documented. For example, progesterone is vital to establishing and maintaining pregnancy and exerts actions on various tissues of the reproductive system. The action of progesterone on tissues outside the reproductive system has been reported but is less well characterized.

Antiprogestins, compounds which inhibit the action of progesterone, have considerable potential for use in the pharmacological regulation of fertility and a variety of conditions and diseases such as breast cancer and endometriosis. The first reported antiprogestin, mifepristone (RU 486), is one of a number of 19-nortestsosterone derivatives with strong affinity for both the progesterone and glucocorticoid receptors and with antiprogestational and antiglucocorticoid activity. A variety of antiprogestins based on the 19-norprogesterone backbone have also been synthesized.

Several drawbacks are associated with the use of known antiprogestins, rendering them less than ideal for chronic administration, particularly when delivered orally. If these and other limitations associated with antiprogestin treatment could be improved, a significant advance in the treatment of hormone-dependent disorders would result.

### SUMMARY OF THE INVENTION

In one embodiment, the present invention provides a capsule suitable for oral administration containing a non-aqueous capsule fill composition comprising an unmicronized selective progesterone receptor modulator (SPRM) in liquid or semi-solid solution with a polyethylene glycol (PEG). Preferably, the SPRM is in a non-micronized crystal form. The capsule may be a gelatin capsule, preferably an immediate release hard gelatin capsule.

In another embodiment, the present invention provides a liquid or semi-solid non-aqueous oral pharmaceutical fill composition for capsule dosage form, said composition comprising, consisting essentially of, or consisting of (a) a pharmaceutically effective amount of an unmicronized selective progesterone receptor modulator (SPRM) per capsule dosage unit (b) polyethylene glycol (PEG) and optionally (c) a preservative, preferably BHT.

In a preferred embodiment, the PEG has an average molecular weight of about 1000 (PEG 1000). In a particularly preferred embodiment, the present invention provides a hard gelatin capsule containing a capsule fill composition consisting of unmicronized CDB-4124 in liquid or semi-solid solution with PEG 1000 and optionally a preservative such as butylated hydroxytoluene (BHT). In a preferred embodiment, the capsule fill composition consists of about 12 mg of unmicronized CDB-4124 in liquid or semi-solid solution with PEG 1000 and optionally BHT. In a particularly preferred embodiment, the capsule fill composition consists of about 4% by weight of CDB-4124, about 95.98% by weight of PEG 1000 and about 0.02% by weight of BHT.

In a related embodiment, a method of making a solid oral dosage form of a pharmaceutical composition comprising an effective amount of an SPRM and a PEG is provided, which method comprises adding a molten solution of SPRM and said PEG to hard gelatin capsules and allowing said molten solution to cool therein, with the proviso that the SPRM is not micronized.

In other embodiments, the present invention provides methods for the treatment of a variety of progesterone related conditions in a patient in need of such treatment by orally administering a hard or soft gelatin capsule containing a non-aqueous capsule fill composition comprising an unmicronized selective progesterone receptor modulator (SPRM) in liquid or semi-solid solution with a polyethylene glycol (PEG) to a patient in need of such treatment.

Progesterone-related conditions that may be treated with the liquid of the invention include, without limitation, endometriosis and pain associated therewith, adenomyosis, endometriomas of the ovary, dysmenorrhea, endocrine hormone-dependent tumors, uterine fibroids, endometrial hyperproliferation, ovarian cancer, cervical cancer and breast cancer. Compositions of the instant invention may also be used to induce menses, to induce labor and for contraception.

### BRIEF DESCRIPTION OF THE DRAWINGS

FIG. 1 illustrates a linear plot of mean plasma concentrations for telapristone (CDB-4124) vs time following oral administration of either of two oral formulations (Formulation A or Formulation B).

FIG. 2 illustrates a semi-log plot of mean plasma concentrations for telapristone vs time for either of two oral formulations (Formulation A or Formulation B).

FIG. 3 illustrates a linear plot of mean plasma concentrations for telapristone metabolite CDB-4453 vs time following oral administration of either of two oral formulations (Formulation A or Formulation B).

FIG. 4 illustrates a semi-log plot of mean plasma concentrations for CDB-4453 vs time for either of two oral formulations (Formulation A or Formulation B).

FIG. 5 illustrates mean and standard deviation for Cₘₐₓ (ng/ml) for telapristone and CDB-4453 for Formulation A and Formulation B.

FIG. 6. Illustrates mean and standard deviation for AUC₀₋₄ for telapristone and CDB-4453 for Formulation A and Formulation B.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

It is to be understood that any ranges, ratios and ranges of ratios that can be formed by any of the numbers or data present herein represent further embodiments of the present invention. This includes ranges that can be formed that do or do not include a finite upper and/or lower boundary. Accordingly, the skilled person will appreciate that many such ratios, ranges and ranges of ratios can be unambiguously derived form the data and numbers presented herein and all represent embodiments of the invention.

Before the present compounds, compositions and methods are disclosed and described, it is to be understood that the terminology used herein is for the purpose of describing particular embodiments only and is not intended to be limiting. It must be noted that, as used in the present specification and the appended claims, the singular forms “a,” “an” and “the” include plural referents unless the context clearly dictates otherwise.

### Definitions

The term “capsule” refers to a hard or soft shell pharmaceutical capsule. The capsule consists of a body and cap and may comprise a fill composition containing a pharmacologically active agent.

The term “oral” administration means that the active agent is in a formulation designed to be ingested, i.e. designed to be delivered to the gastrointestinal system for absorption.

The term “effective dosage” means an amount of the composition's active component sufficient to treat a particular condition.

The term “selective progesterone receptor modulators” means compounds that affect functions of progesterone receptor in a tissue-specific manner. The compounds act as progesterone receptor antagonists in some tissues (for example, in breast tissue) and as progesterone receptor agonists in other tissues (for example, in the uterus).

The term “treat” or “treatment” as used herein refers to any treatment of any progesterone-dependent disorder or disease, and includes, but is not limited to, inhibiting the disorder or disease arresting the development of the disorder or disease; relieving the disorder or disease, for example, causing regression of the disorder or disease; or relieving the condition caused by the disease or disorder, relieving the symptoms of the disease or disorder.

The term “prevent” or “prevention,” in relation to a progesterone-dependent disorder or disease, means preventing the onset of disorder or disease development if none had occurred, or preventing further disorder or disease development if the disorder or disease was already present. For example, compositions of the present invention may be used to prevent the recurrence of tumors. Recurrence of tumors may occur because of residual microscopic groups or nests of tumor cells which subsequently expand into clinically detectable tumors.

The term “progesterone agonist” means a compound that binds to a progesterone receptor and mimics the action of the natural hormone.

The term “progesterone antagonist” means a compound that binds to a progesterone receptor and inhibits the effect of progesterone.

SPRMs such as CDB-4124 (Proellex; telapristone; 21-methoxy-17a-acetoxy-11ß-(4 N, N-dimethylaminophenyl)-19-norpregna-4,9-diene-3,20-dione) are hydrophobic and costly to synthesize. Micronization of these drugs improves solubility and bioavailability. Unfortunately, micronization of CDB-4124 is a time consuming and expensive endeavor and moreover results in a significant loss of the active agent. Further, an inherent drawback of micronization is that the material obtained must comply with stringent particle size specifications and the filling of hard gelatin capsules with micronized powder is a difficult operation, particularly if weight variation homogeneity is considered.

The present inventors have surprisingly discovered that the present composition avoids the need for micronizing the SPRM, while providing a bioavailability comparable and even favorable to that afforded by conventional SPRM compositions which employ micronization when administered orally. Compared to a fill composition in which CDB-4124 is micronized, the present fill composition has a lower Cₘₐₓ and higher AUC_{0-∞} when administered orally. Reduction of Cₘₐₓ while maintaining exposure of the active agent has important safety advantages since liver toxicity is concentration dependent for SPRMs such as CDB-4124 and it has previously been shown that Cₘₐₓ is most relevant due to higher concentrations of the drug leading to the formation of stable adducts to liver proteins.

In several embodiments, the present invention relates to a non-aqueous liquid or semi-liquid oral pharmaceutical fill composition for capsule dosage form, said composition consisting essentially of or consisting of an unmicronized SPRM and a PEG. In preferred embodiments, the SPRM is CDB-4124 and the PEG is PEG 1000. In particularly preferred embodiments, the capsule fill composition consists of or consists essentially of about 12 mg of unmicronized CDB-4124 and about 100% w/w PEG 1000 as excipient. Optionally, butylated hydroxytoluene is also present at 0.02% excipient w/w (as antioxidant)

In some embodiments, the fill composition is essentially free of, or does not comprise, polyglycolized glyercide, representative examples of which include, but are not limited to GELUCIRE 44/14®, LABRAFIL® AND LABRASOL®. In related embodiments, the fill composition also does not comprise PECEOL®.

In some embodiments, the capsule fill composition does not comprise polyvinyl pyrrolidone.

Polyethylene glycols are addition polymers of ethylene oxide and water usually designated by a number roughly corresponding to molecular weight. PEGs below 700 molecular weight occur as liquids PEGs between 700-1000 are semi-solid. PEGs over 1000 are waxy solids, flakes or free-flowing powders. In some embodiments, the polyethylene glycol has a molecular weight range of about 200 to about 1000, preferably from about 700 to about 1000. In a preferred embodiment, the polyethylene glycol is PEG 1000.

The fill composition may be prepared in a conventional manner, for example, by a method comprising heating the PEG; adding an effective amount of an unmicronized SPRM and further mixing until all or substantially all of the SPRM is solubilized, e.g. until the solution is visually clear. This method of preparing the composition constitutes another aspect of the present invention.

The resulting fill composition is then formulated into hard or soft gelatin capsules by well-known manufacturing technology.

One embodiment is directed to a soft gelatin capsule comprising a non-aqueous liquid or semi-liquid oral pharmaceutical fill composition comprising, consisting essentially of or consisting of an umicronized SPRM, preferably CDB-4124, and a PEG, preferably PEG 1000. Soft gelatin capsule shells can be produced according to any of the acceptable methods known in the art for production of such capsules. Soft gelatin shells may comprise from about 20% to about 80% gelatin. The gelatin can be of Type A, Type B, or a mixture thereof. The soft gelatin shells may also comprise a plasticizer. Suitable plasticizers include glycerin, xylitol, sorbitol, polyglycyerol, non-crystallizing solutions of sorbitol, glucose, fructose, glucose syrups, or mixtures thereof. Preferably, the plasticizer is glycerin. The soft gelatin shells may also comprise water and other ingredients, such as taste modifiers, coloring agents, preservatives and moisture retaining agents.

Another embodiment is directed to a hard gelatin capsule comprising a non-aqueous liquid or semi-liquid oral pharmaceutical fill composition comprising, consisting essentially of or consisting of an umicronized SPRM, preferably CDB-4124, and a PEG, preferably PEG 1000. Hard gelatin capsules for liquid filling have usually a volume of from 0.3 to 1.0 ml. The available volume is in general from 85-95 vol-%. In comparison to soft gelatin capsules, the moisture uptake of hard gelatin capsules is usually much lower than of soft gelatin capsules. In the hard capsule encapsulation process the capsule is usually pre-fabricated and supplied empty, whereas in the soft gelatin capsule the encapsulation and filling take place simultaneously. Various methods can be used to seal the hard gelatin capsules according to the invention. Preferred methods are banding using a gelatin band and sealing using a hydroalcoholic solution.

SPRMs

The capsule fill composition comprises an amount of an SPRM which has a therapeutic effect when a capsule containing the capsule fill composition is administered orally to a subject, preferably a human female.

In one embodiment, the capsule fill composition comprises a steroid compound disclosed in U.S. Pat. Nos. 6,861,415 and 6,900,193, the contents of which are incorporated herein by reference. In a preferred embodiment, the steroid compound is CDB-4124 (21-methoxy-17a-acetoxy-11β-(4 N, N-dimethylaminophenyl)-19-norpregna-4,9-diene-3,20-dione) or CDB-4453 (21-methoxy-17α-acetoxy-11β-(4-N-methylaminophenyl)-19-norpregna-4,9-diene-3,20-dione).

In other embodiments, the capsule fill composition comprises an SPRM such as, without limitation, Mifepristone (RU-486; 11β-[4 N,N-dimethylaminophenyl]-17β-hydroxy-17-(1-propynyl)-estra-4,9-dien-3-one), Lilopristone (11β-(4 N,N-dimethylaminophenyl)-17β-hydroxy-17-((Z)-3-hydroxypropenyl)estra-4,9-dien-3-one), Onapristone (11β-(4 N,N-dimethylaminophenyl)-17α-hydroxy-17-(3-hydroxypropyl)-13α-estra-4,9-dien-3-one), asoprisnil (benzaldehyde, 4-[(11β,17β)-17-methoxy-17-(methoxymethyl)-3-oxoestra-4,9-dien-11-yl]-1-(E)-oxim; J867/asoprisnil), its metabolite J912 (4-[17β-Hydroxy-17α-(methoxymethyl)-3-oxoestra-4,9-dien-11β-yl]benzaldehyd-(1E)-oxim), CDB-2914 (17α-acetoxy-11β-(4-N,N-dimethylaminophenyl)-19-norpregna-4,9-dien-3,20-dione) or vilaprisan ((8S,11R,13S,14S,17R)-17-hydroxy-13-methyl-11-(4-(methyl sulfonyl)phenyl)-17-(perfluoroethyl)-1,2,6,7,8,11,12,13,14,15,16,17-dodecahydro-3H-cyclopenta[a]phenanthren-3-one).

In related aspects, capsule fill compositions comprise a pharmaceutically acceptable salt of an SPRM. Depending on the process conditions the salt compound obtained may be either in neutral or salt form. Salt forms include hydrates and other solvates and also crystalline polymorphs. Both the free base and the salts of these end products may be used in accordance with the invention. Acid addition salts may in a manner known per se be transformed into the free base using basic agents such as alkali or by ion exchange. The free base obtained may also form salts with organic or inorganic acids.

In the preparation of acid addition salts, preferably such acids are used which form suitably pharmaceutically acceptable salts. Examples of such acids are hydrochloric acid, sulfuric acid, phosphoric acid, nitric acid, aliphatic acid, alicyclic carboxylic or sulfonic acids, such as formic acid, acetic acid, propionic acid, succinic acid, glycolic acid, lactic acid, malic acid, tartaric acid, citric acid, ascorbic acid, glucuronic acid, fumaric acid, maleic acid, hydroxymaleic acid, pyruvic acid, aspartic acid, glutamic acid, p-hydroxybenzoic acid, embonic acid, ethanesulfonic acid, hydroxyethanesulfonic acid, phenylacetic acid, mandelic acid, alogenbensenesulfonic acid, toluenesulfonic acid, galactaric acid, galacturonic acid or naphthalenesulfonic acid. All crystalline form polymorphs may be used in accordance with the invention. A preferred salt of an SPRM is telapristone acetate.

Base addition salts may also be used in accordance with the invention and may be prepared by contacting the free acid form with a sufficient amount of the desired base to produce the salt in the conventional manner. The free acid form may be regenerated by contacting the salt form with an acid and isolating the free acid in the conventional manner. Pharmaceutically acceptable base addition salts are formed with metals or amines, such as alkali and alkali earth metals or organic amines. Examples of metals used as cations are sodium, potassium, calcium, magnesium and the like. Examples of suitable amines are amino acids such as lysine, choline, diethanolamine, ethylenediamine, N-methylglucamine and the like.

Disorders that May be Treated by Orally Administering Capsules Containing a Non-Aqueous Fill Composition as Described Herein

Capsules containing a non-aqueous capsule fill composition comprising an unmicronized SPRM in liquid or semi-solid solution with a PEG may be administered orally to a subject to treat a variety of disorders or achieve a variety of desired therapeutic results in a subject. Preferably the subject is a female mammal, most preferably a human female.

In some embodiments of the invention, the capsule is orally administered to a female patient in need thereof in order to treat a disorder selected from the group consisting of endometrial hyperproliferation, endometriosis (or pain associated therewith), dysmenorrhea, uterine fibroids, adenomyosis, endometrioma, ovarian cancer, cervical cancer. In a preferred embodiment, endometriosis, dysmennorhea, uterine fibroids, adenomyosis, ovarian cancer or cervical cancer is treated by orally administering the capsule to a patient in need of such treating.

In another embodiment of the invention, the capsule is orally administered to a female in need thereof in order to induce menses in the female.

In yet another embodiment of the invention, the capsule is orally administered to a female in need thereof in order to induce labor.

Dosages and Administration Regimens

A therapeutically effective amount of an SPRM required for use in therapy varies with the length of time that activity is desired, and the age and the condition of the patient to be treated, among other factors, and is ultimately determined by the attendant physician. In general, however, doses employed for human treatment typically are in the range of about 0.001 mg/kg to about 500 mg/kg per day, for example about 1 μg/kg to about 1 mg/kg per day or about 1 μg/kg to about 100 μg/kg per day. For most large mammals, the total daily dosage is from about 1 to 100 mg, preferably from about 2 to 80 mg. The dosage regimen may be adjusted to provide the optimal therapeutic response. The desired dose may be conveniently administered in a single dose, or as multiple doses administered at appropriate intervals, for example as two, three, four or more subdoses per day. In some embodiments, a liquid or semi-solid non-aqueous oral pharmaceutical fill composition for capsule dosage form as described herein comprises CDB-4124 present in an amount of about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, or about 25 mg per capsule. In a particularly preferred embodiment, a hard gelatin capsule containing a capsule fill composition consisting of about 12 mg of CDB-4124, PEG 1000 and optionally a preservative such as BHT is administered once per day to a human female in need thereof.

Illustratively, a capsule of the invention may be orally administered to a subject to provide the subject with an SPRM in an amount of about 1 μg/kg to about 1 mg/kg body weight, for example about 1 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg or about 1 mg/kg body weight

Gelatin capsules comprising a fill composition comprising, consisting essentially of or consisting of an SPRM and PEG 1000, are suitable for prolonged/chronic oral administration because the composition exhibits a relatively low Cₘₐₓ and therefore is expected to avoid liver toxicity. In one embodiment, the a capsule of the invention is administered for an administration period of least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. The capsules may also be administered for an administration period of least 1, 2, 3, 4, 5, 6, 7, 8, 9, 10, 11, 12 or more months. The capsules may also be administered for an administration period of at least 1, 2, 3, 4, 5, 6, 7, 8, 9, 10 or more years. During the administration period, the capsules may be administered daily or periodically such as every other day, every other month, and the like but are preferably administered once per day. The capsules may also be administered intermittently. For example, the capsules may be administered for an administration period of 1, 2, 3, 4, 5 or more months, followed by a period of discontinuance, followed by an administration period of 1, 2, 3, 4, 5 or more months, and so on.

In one embodiment, the capsule is administered intermittently such that the subject undergoes menses during at least one discontinuance period. This approach is expected to avoid the adverse effects associated with a thickened or stagnant endometrium that may accompany extended treatment with progesterone antagonists, such as spotting, breakthrough bleeding, endometrial hyperproliferation or endometrial cancer. At least one, and preferably every discontinuance period is of sufficient length for the subject to experience menstruation. More preferably, the subject experiences menstruation during every discontinuance period. In a particularly preferred embodiment, the capsule is administered daily for an administration period of about 18 weeks, followed by a discontinuance period (off drug interval) during which the subject experiences menstruation, followed by another administration period of about 16 or 18 weeks and so on. The inventors have determined that orally administering a gelatin capsule containing a non-aqueous capsule fill composition consisting of 12 mg of unmicronized CDB-4124, PEG 1000 and BHT once per day to human females with uterine fibroids resulted in 100% induction of amenorrhea after two 18 week cycles and reduced uterine fibroids by an average of 64.9%.

Patients undergoing treatments with the compositions of the instant invention should be monitored routinely for their serum estrogen and glucocorticoid levels.

The following non-limiting examples are provided to aid in understanding the teachings of the instant invention.

### Example 1. Double-Blind Crossover Study in Healthy Volunteers to Compare Two Formulations of Proellex for Oral Administration

Methods

A Phase I study was initiated to compare the pharmacokinetics after a single oral administration of either of two oral formulations containing 12 mg of Proellex (telapristone acetate/CDB-4124) in healthy female volunteers. Formulation A was a dry-filled gelatin capsule containing 10.5% of micronized CDB-4124, 87.6% microcrystalline cellulose, and 1.9% magnesium stearate. Formulation B was a liquid-filled gelatin capsule containing 4.0% unmicronized CDB-4124, 95.98% polyethylene glycol (PEG) 1000, and 0.02% butylated hydroxytoluene.

In the first treatment period, twelve healthy human females were randomly assigned to receive either a single dose of Formulation A or single dose of Formulation B. After a 7-day washout period, the subjects received the alternate treatment in the second period. The two formulations were provided as capsules containing 12 mg Proellex. The study drugs were administered orally with 240 mL of water.

Blood Sample Collection and Processing

Blood samples for pharmacokinetic profiles were collected on the days that the single doses of Formulations A and B were administered, Days 0 and 8. For each profile, blood samples (4 mL) were collected pre-dose and at 0.5, 1, 2, 3, 4, 8, 12, 16, 24, 48, and 72 hours post-dose. Lithium heparin was used as an anticoagulant. After collection, blood samples were mixed by inversion and centrifuged for 10 minutes at 400×g and 4 C. Plasma was collected and centrifuged for 25 minutes at 900×g and 4 C to remove platelets. The platelet-free plasma supernatant was used for analysis.

Analysis of Telapristone and CDB-4453 Concentrations

The concentrations of telapristone (CDB-4124) and CDB-4453 (primary metabolite of telapristone) in plasma were determined using a validated LC-MS/MS method. The calibration ranges for both telapristone and CDB-4453 were 1.00 to 100 ng/mL, and the lower limit of quantitation was 1.00 ng/mL for both analytes.

Results and Discussion

Plasma Concentrations of Telapristone and CDB-4453

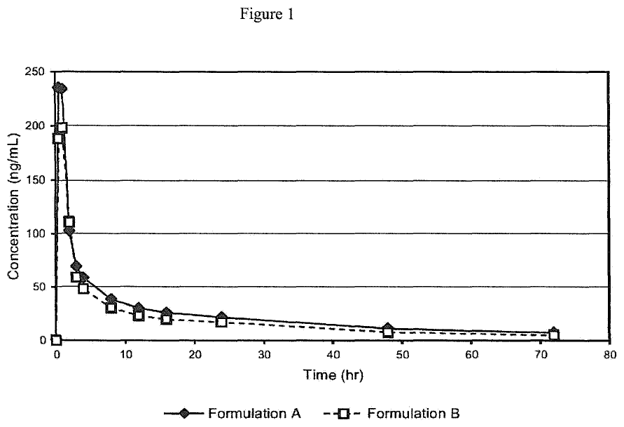

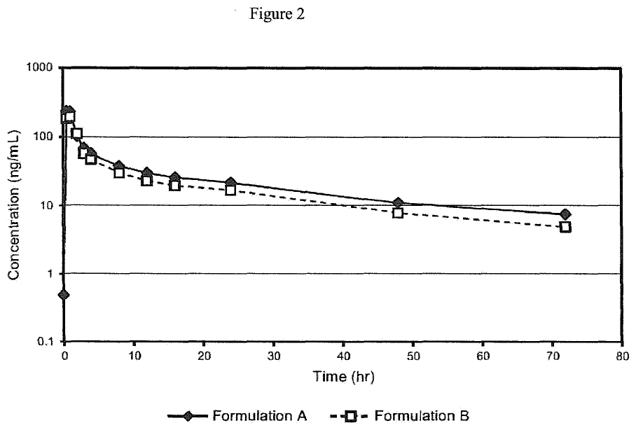

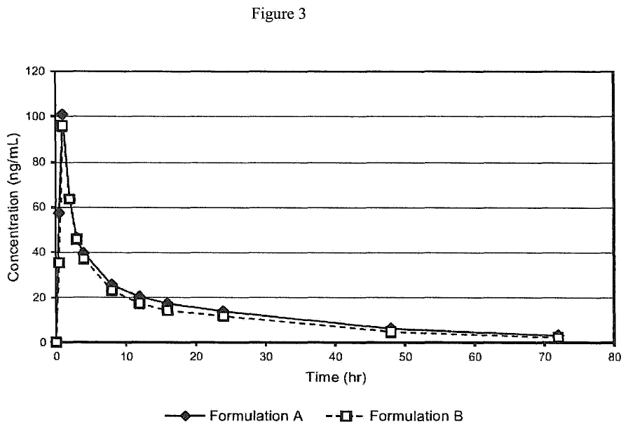

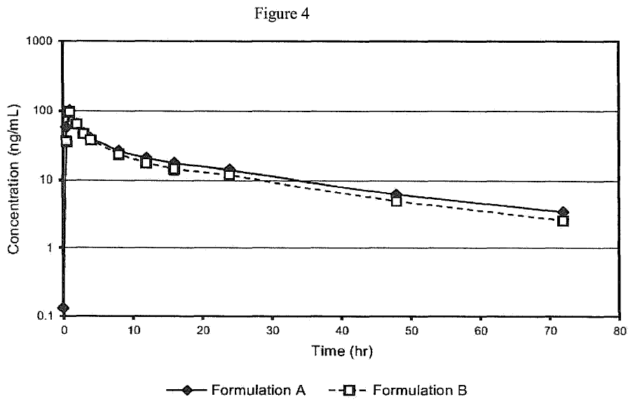

The mean concentrations and standard deviations for telapristone and CDB-4453 are shown in Tables 1 and 2, respectively, below. FIGS. 1-4 show linear and semi-log plots of the mean concentrations of telapristone and CDB-4453. The mean concentrations of telapristone and CDB-4453 were very similar for the two formulations.

TABLE 1
Mean Plasma Concentrations and Standard Deviations for Telapristone

| | Plasma Concentration (ng/mL) | |
|---|---|---|
| Time | Formulation A | Formulation B |

| (hr) | Mean ± SD | % CV | Mean ± SD | % CV |
|---|---|---|---|---|
| 0 | 0.488 ± 1.692 | 346 | 0 | |
| 0.5 | 235 ± 258 | 110 | 189 ± 151 | 79.9 |
| 1 | 234 ± 146 | 62.2 | 198 ± 103 | 52.1 |
| 2 | 103 ± 60 | 58.7 | 111 ± 70 | 63.4 |
| 3 | 69.0 ± 40.0 | 58.0 | 58.9 ± 32.2 | 54.6 |
| 4 | 58.6 ± 36.4 | 62.2 | 48.2 ± 27.4 | 56.8 |
| 8 | 38.8 ± 28.9 | 74.5 | 30.5 ± 19.1 | 62.8 |
| 12 | 30.2 ± 23.1 | 76.6 | 23.2 ± 16.6 | 71.7 |
| 16 | 26.1 ± 22.6 | 86.3 | 19.9 ± 14.2 | 71.1 |
| 24 | 21.8 ± 21.9 | 100 | 17.0 ± 14.5 | 85.2 |
| 48 | 11.0 ± 14.7 | 134 | 7.80 ± 8.93 | 114 |
| 72 | 7.44 ± 13.08 | 176 | 4.91 ± 7.70 | 157 |

n = 12 in all cases. Standard deviation values were not calculated when all values were 0 ng/mL.

TABLE 2
Mean Plasma Concentrations and Standard Deviations for CDB-4453

| | Plasma Concentration (ng/mL) | |
|---|---|---|
| Time | Formulation A | Formulation B |

| (hr) | Mean ± SD | % CV | Mean ± SD | % CV |
|---|---|---|---|---|
| 0 | 0.132 ± 0.456 | 346 | 0 | |
| 0.5 | 57.4 ± 66.8 | 117 | 35.3 ± 42.5 | 120 |
| 1 | 101 ± 43 | 42.6 | 95.9 ± 61.7 | 64.4 |
| 2 | 63.9 ± 23.9 | 37.3 | 63.7 ± 27.8 | 43.6 |
| 3 | 46.3 ± 15.5 | 33.5 | 45.8 ± 30.5 | 66.6 |
| 4 | 39.5 ± 13.5 | 34.1 | 37.0 ± 18.9 | 51.0 |
| 8 | 25.8 ± 9.3 | 36.1 | 23.3 ± 11.3 | 48.5 |
| 12 | 20.5 ± 8.5 | 41.3 | 17.5 ± 8.4 | 48.0 |
| 16 | 17.5 ± 8.3 | 47.4 | 14.5 ± 7.2 | 49.6 |
| 24 | 14.0 ± 7.6 | 53.9 | 11.9 ± 7.1 | 59.3 |
| 48 | 6.27 ± 4.14 | 66.0 | 4.94 ± 3.35 | 67.8 |
| 72 | 3.39 ± 3.15 | 92.9 | 2.53 ± 2.00 | 78.9 |

n = 12 in all cases. Standard deviation values were not calculated when all values were 0 ng/mL.

Pharmacokinetic Parameters for Telapristone and CDB-4453

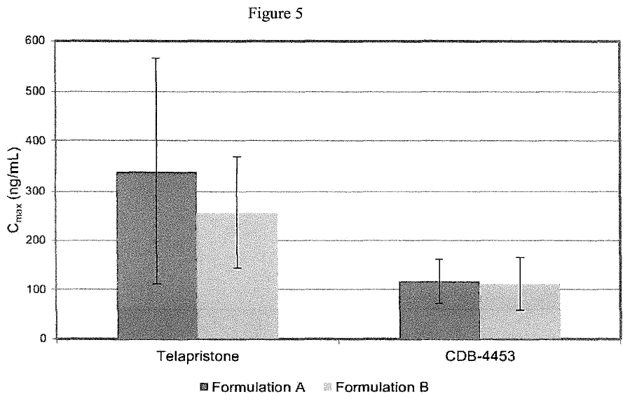

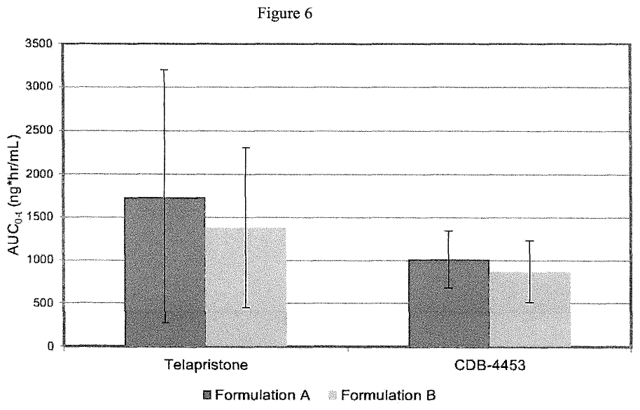

The descriptive statistics for the parameters for telapristone and CDB-4453 are in Tables 3 and 4, respectively, below. FIGS. 5 and 6 show the mean concentrations and standard deviations for Cₘₐₓ and AUC0-t, respectively, for telapristone and CDB-4453. The results of the bioequivalence comparisons are in Table 5.

TABLE 3
Descriptive Statistics for Pharmacokinetic Parameters for Telapristone

| | | | | Geometric | | | |
|---|---|---|---|---|---|---|---|
| Parameter | Formulation | Mean ± SD | % CV | Mean | Median | Range | n |
| Cₘₐₓ | A | 338 ± 228 | 67.4 | 272 | 264 | 73.0-805 | 12 |
| (ng/mL) | B | 256 ± 113 | 44.2 | 222 | 249 | 35.4-455 | 12 |
| Tₘₐₓ | A | 0.873 ± 0.426 | 48.8 | — | 0.975 | 0.417-1.92 | 12 |
| (hr) | B | 0.916 ± 0.570 | 62.2 | — | 0.740 | 0.417-2.02 | 12 |
| AUC₀₋₂₄ | A | 1.133 ± 734 | 64.8 | 946 | 857 | 328-2,791 | 12 |
| (ng * hr/mL) | B | 935 ± 492 | 52.6 | 817 | 857 | 310-1,841 | 12 |
| AUC₀₋ₜ | A | 1,738 ± 1,462 | 84.1 | 1,291 | 1,204 | 324-4,977 | 12 |
| (ng * hr/mL) | B | 1,377 ± 925 | 67.2 | 1,127 | 1,107 | 464-2,985 | 12 |
| AUC_{0-∞} | A | 1,251 ± 757 | 60.5 | 1,063 | 1,172 | 329-2,984 | 10 |
| (ng * hr/mL) | B | 1,341 ± 952 | 71.0 | 1,102 | 997 | 473-3,200 | 11 |
| λ_{z} | A | 0.0597 ± 0.0581 | 97.3 | 0.0436 | 0.0428 | 0.00948-0.224 | 12 |
| (hr⁻¹) | B | 0.0521 ± 0.0385 | 74.0 | 0.0415 | 0.0407 | 0.00910-0.145 | 12 |
| t_{1/2} | A | 21.4 ± 19.0 | 89.0 | 15.9 | 16.2 | 3.09-73.1 | 12 |
| (hr) | B | 21.4 ± 18.6 | 87.1 | 16.7 | 17.2 | 4.79-76.2 | 12 |

TABLE 4
Descriptive Statistics for Pharmacokinetic Parameters for CDB-4453

| | | | | Geometric | | | |
|---|---|---|---|---|---|---|---|
| Parameter | Formulation | Mean ± SD | % CV | Mean | Median | Range | n |
| Cₘₐₓ | A | 117 ± 45 | 38.2 | 109 | 122 | 48.5-200 | 12 |
| (ng/mL) | B | 111 ± 55 | 49.2 | 91.6 | 121 | 11.2-205 | 12 |
| Tₘₐₓ | A | 1.05 ± 0.52 | 49.6 | — | 0.975 | 0.467-2.17 | 12 |
| (hr) | B | 1.26 ± 0.70 | 55.7 | — | 1.06 | 0.433-2.92 | 12 |
| AUC₀₋₂₄ | A | 659 ± 182 | 27.7 | 637 | 628 | 418-994 | 12 |
| (ng * hr/mL) | B | 588 ± 243 | 41.3 | 539 | 563 | 184-1,075 | 12 |
| AUC₀₋ₜ | A | 1,013 ± 332 | 32.8 | 956 | 1,022 | 499-1,428 | 12 |
| (ng * hr/mL) | B | 869 ± 357 | 41.0 | 806 | 804 | 393-1,533 | 12 |
| AUC_{0-∞} | A | 1,117 ± 410 | 36.7 | 1,038 | 1,056 | 524-1,650 | 11 |
| (ng * hr/mL) | B | 957 ± 380 | 39.7 | 890 | 925 | 411-1,637 | 11 |
| λ_{z} | A | 0.0561 ± 0.0552 | 98.3 | 0.0407 | 0.0396 | 0.00922-0.211 | 12 |
| (hr⁻¹) | B | 0.0507 ± 0.0425 | 84.0 | 0.0396 | 0.0348 | 0.0108-0.162 | 12 |
| t_{1/2} | A | 22.5 ± 18.9 | 83.9 | 17.0 | 17.7 | 3.29-75.2 | 12 |
| (hr) | B | 21.7 ± 15.3 | 70.8 | 17.5 | 20.3 | 4.28-63.9 | 12 |

TABLE 5
Comparison of Least Squares Mean Values for Formulation B versus Formulation A

| | | Least Squares | | Back | | Ratio LSM | 90% Confidence | | |
|---|---|---|---|---|---|---|---|---|---|
| | | Mean (LSM) | | Transformed LSM | | Formulations | Interval | | |
| | | Test: | Reference: | Test: | Reference: | B/A | Lower | Upper | p- |
| Analyte | Parameter | Formulation B | Formulation A | Formulation B | Formulation A | (%) | (%) | (%) | value |
| Telapristone | Ln(Cₘₐₓ) | 5.40 | 5.61 | 222 | 272 | 81.6 | 54.7 | 122 | 0.378 |
| | Ln(AUC₀₋₂₄) | 6.71 | 6.85 | 817 | 946 | 86.4 | 70.9 | 105 | 0.210 |
| | Ln(AUC₀₋ₜ) | 7.03 | 7.16 | 1,127 | 1,291 | 87.4 | 73.9 | 103 | 0.175 |
| | Ln(AUC_{0-∞})^{a} | 6.93 | 7.00 | 1,019 | 1,096 | 93.0 | 77.0 | 112 | 0.497 |
| | Ln(λ_{z}) | −3.18 | −3.13 | 0.0415 | 0.0436 | 95.3 | 83.1 | 109 | 0.542 |
| | Tₘₐₓ | 0.916 | 0.873 | — | — | 105 | 55.8 | 154 | 0.858 |
| | t_{1/2} | 21.4 | 21.4 | — | — | 100 | 84.7 | 115 | 0.991 |
| CDB-4453 | Ln(Cₘₐₓ) | 4.52 | 4.69 | 91.6 | 109 | 84.2 | 55.7 | 127 | 0.470 |
| | Ln(AUC₀₋₂₄) | 6.29 | 6.46 | 539 | 637 | 84.6 | 66.8 | 107 | 0.229 |
| | Ln(AUC₀₋ₜ) | 6.69 | 6.86 | 806 | 956 | 84.4 | 70.6 | 101 | 0.116 |
| | Ln(AUC_{0-∞})^{b} | 6.79 | 6.95 | 888 | 1,040 | 85.3 | 70.2 | 104 | 0.170 |
| | Ln(λ_{z}) | −3.23 | −3.20 | 0.0396 | 0.0407 | 97.1 | 86.6 | 109 | 0.652 |
| | Tₘₐₓ | 1.26 | 1.05 | — | — | 120 | 71.7 | 169 | 0.466 |
| | t_{1/2} | 21.7 | 22.5 | — | — | 96.2 | 82.7 | 110 | 0.620 |

^{a}Subjects 101-01-008 and 101-01-014 were not included in the analysis for AUC_{0-∞} due to lack of reliable values for one or both formulations.
^{b}Subject 101-01-008 was not included in the analysis for AUC_{0-∞} due to lack of reliable values for both formulations.

For telapristone, the mean, geometric mean, and median values for Cₘₐₓ, AUC₀₋₂₄, and AUC₀₋ₜ were similar for Formulations A and B, with the values being very slightly lower, but less variable for Formulation B, as shown by the standard deviations and ranges (Table 3, FIGS. 5 and 6). Reliable values for AUC_{0-∞} could not be calculated for two subjects for Formulation A and for one subject for Formulation B (Appendix B). The resulting mean AUC_{0-∞} was slightly higher for Formulation B than for Formulation A. The LSM ratio for Cₘₐₓ was 81.6% with a 90% CI of 54.7% to 122% (Table 5). The LSM ratio for AUC₀₋₄ was 87.4% with a 90% CI of 73.9% to 103%. These results indicate that the exposure to telapristone from Formulation B was within 20% of the exposure from Formulation A; however, the 90% CI values were not within 80% to 125%. The LSM results for AUC₀₋₂₄ were similar to those for AUC₀₋ₜ; however, since AUC₀₋₂₄ is a partial area, it not as good a measure of exposure as AUC₀₋ₜ. The LSM results for AUG_{0-∞} indicated slightly more equal exposure, 93.0% with 90% CI values 77.0% to 112%. However, since there missing values for AUC_{0-∞}, it is not as representative of the study population as AUC₀₋ₜ. In spite of a moderately large intersubject variability for λ_{z} and t_{1/2} with % CV values of 74% or greater, the mean values were nearly identical (Table 3), and the LSM ratios were close to 100% with 90% CI values between 80% and 125% indicating that the elimination rates were the same for both formulations (Table 5). The median Tₘₐₓ values were slightly less than 1 hour for both formulations, indicating rapid absorption (Table 3). The LSM ratio for Tₘₐₓ was 105% with a 90% CI range of 55.8% to 154% (Table 5). There were no statistically significant differences (p>0.05) for any of the parameters for telapristone, and the 90% CI intervals included 100% in all cases (Table 5).

For the metabolite CDB-4453, the mean, geometric mean, and median values for Cₘₐₓ were nearly identical for Formulations A and B (Table 4). The mean, geometric mean, and median values for AUC₀₋₂₄ and AUC₀₋ₜ were similar for Formulations A and B, with the values being slightly lower with similar variability (Table 4, FIGS. 5 and 6). Reliable values for AUC_{0-∞} could not be calculated for either Formulation A or B for one subject. The resulting mean, geometric mean, and median AUC_{0-∞} values for the other 11 subjects were very similar to the corresponding values for AUC₀₋ₜ with AUC_{0-∞} being very slightly lower for Formulation B than for Formulation A. The LSM ratio for Cₘₐₓ was 84.2% with a 90% CI of 55.7% to 127% (Table 5). The LSM ratio for AUC₀₋ₜ was 84.4% with a 90% CI of 70.6% to 101%. These results indicate that the exposure to telapristone from Formulation B was within 20% of the exposure from Formulation A; however, the 90% CI values were not within 80% to 125%. The LSM results for AUC₀₋₂₄ and AUC_{0-∞} were similar to those for AUC₀₋ₜ, however, these parameters not as good as measures of exposure as AUC₀₋ₜ since AUC₀₋₂₄ is a partial area and there were some missing values for AUC_{0-∞}. In spite of a moderately large intersubject variability for λ_{z} and t_{1/2} with % CV values of 71% or greater, the mean values were very similar (Table 4), and the LSM ratios were close to 100% with 90% CI values between 80% and 125% indicating that the elimination rates were the same for both formulations (Table 5). The median Tₘₐₓ values were close to 1 hour for both formulations, indicating rapid absorption and metabolism of telapristone to CDB-4453 (Table 4). The LSM ratio for Tₘₐₓ was 120% with a 90% CI range of 71.7% to 169% (Table 5). There were no statistically significant differences (p>0.05) for any of the parameters for CDB-4453, and the 90% CI intervals included 100% in all cases (Table 5).

Conclusions

The exposure to telapristone and CDB-4453 was similar after administration of single doses of Formulation A and Formulation B. For the primary measures of exposure, Cₘₐₓ and AUC₀₋ₜ, the ratios of the LSM values for Formulation B/Formulation A were between 80% and 90%. Although the 90% CI ranges for Cₘₐₓ and AUC₀₋ₜ were not included within 80% to 125%, there were no statistically significant differences between the formulations and the 90% CI ranges included 100% in all cases. The results for AUC₀₋₂₄ and AUC_{0-∞} were similar to the results for AUC₀₋ₜ. Median Tₘₐₓ occurred at slightly less than 1 hour for telapristone and very close to 1 hour for CDB-4453 for both formulations, indicating rapid absorption of telapristone and rapid metabolism to CDB-4453. In spite of a moderately large intersubject variability for λ_{z} and t_{1/2}, the mean values for both telapristone and CDB-4453 were very similar, and the LSM ratios were close to 100% with 90% CI values between 80% and 125% indicating that the elimination rates were the same for both formulations.

A Synopsis of the pharmacokinetic parameters and bioequivalence LSM ratios is provided below at Tables 6 and 7 respectively.

TABLE 6

| | Telapristone | | CDB-4453 | |
|---|---|---|---|---|
| | (mean ± SD) | | (mean ± SD) | |
| Parameter | Formulation A | Formulation B | Formulation A | Formulation B |
| Cₘₐₓ (ng/mL) | 338 ± 228 | 256 ± 113 | 117 ± 45 | 111 ± 55 |
| Tₘₐₓ (hr)^{a} | 0.975 | 0.740 | 0.975 | 1.06 |
| | (0.417-1.92)^{a} | (0.417-2.02)^{a} | (0.467-2.17)^{a} | (0.433-2.92)^{a} |
| AUC₀₋₂₄ (ng * hr/mL) | 1,133 ± 734 | 935 ± 492 | 659 ± 182 | 588 ± 243 |
| AUC₀₋ₜ (ng * hr/mL) | 1,738 ± 1,462 | 1,377 ± 925 | 1,013 ± 332 | 869 ± 357 |
| AUC_{0-∞} (ng * hr/mL) | 1,251 ± 757 | 1,341 ± 952 | 1,117 ± 410 | 957 ± 380 |
| λ_{z} (hr⁻¹) | 0.0597 ± 0.0581 | 0.0521 ± 0.0385 | 0.0561 ± 0.0552 | 0.0507 ± 0.0425 |
| t_{1/2} (hr) | 21.4 ± 19.0 | 21.4 ± 18.6 | 22.5 ± 18.9 | 21.7 ± 15.5 |

^{a}Values for Tₘₐₓ presented as median with range.

TABLE 7

| Analyte | Parameter | Ratio | 90% Cl | p-value |
|---|---|---|---|---|
| Telapristone | Ln (Cₘₐₓ) | 81.6% | 54.7%-122% | 0.378 |
| | Ln (AUC₀₋₂₄) | 86.4% | 70.9%-105% | 0.210 |
| | Ln (AUC₀₋ₜ) | 87.4% | 73.9%-103% | 0.175 |
| | Ln (AUC_{0-∞}) | 93.0% | 77.0%-112% | 0.497 |
| | Ln (λ_{z}) | 95.3% | 83.1%-109% | 0.542 |
| | Tₘₐₓ | 105% | 55.8%-154% | 0.858 |
| | t_{1/2} | 100% | 84.7%-115% | 0.991 |
| CDB-4453 | Ln (Cₘₐₓ) | 84.2% | 55.7%-127% | 0.470 |
| | Ln (AUC₀₋₂₄) | 84.6% | 66.8%-107% | 0.229 |
| | Ln (AUC₀₋ₜ) | 84.4% | 70.6%-101% | 0.116 |
| | Ln (AUC_{0-∞}) | 85.3% | 70.2%-104% | 0.170 |
| | Ln (λ_{z}) | 97.1% | 86.6%-109% | 0.652 |
| | Tₘₐₓ | 120% | 71.7%-169% | 0.466 |
| | t_{1/2} | 96.2% | 82.7%-110% | 0.620 |

### Example 2: Clinical Effects of Orally Administered Formulation B

A Phase 2 study was initiated to test the effects of Formulation B orally administered to women with confirmed uterine fibroids by MRI at baseline who were experiencing more than 80 mL of blood loss during menses as confirmed by alkaline hematin assessment. Gelatin capsules, containing a fill composition consisting of 12 mg of unmicronized CDB-4124 and PEG 1000 (and BHT as a preservative; Formulation B of Example 1) were administered once per day to women with uterine fibroids for 18 weeks of blinded treatment and were then withdrawn from the medication to allow for menses. After menses occurred, a second 18 week course of treatment followed.

Substantial and statistically significant reductions in excessive menstrual bleeding, the key symptom of uterine fibroids and the primary endpoint of the studies, was observed. Amenorrhea, cessation of menses, is known to occur when a sufficiently high plasma concentration of Proellex® is achieved. The incidence of amenorrhea in active treatment groups consistently showed a statistically significantly difference from the rate in placebo-treated subjects. At the end of the second course of treatment (36 weeks total active treatment), 100% of subjects achieved amenorrhea (compared to only 50% of vaginally treated subjects). The oral dosage form provided for consistent suppression of menses.

Along with changes in menstrual patterns, fibroid size, measured by MM, was reduced in volume. A median 64.9% reduction in fibroid size compared to baseline was achieved after two 18 week cycles and was statistically different from the change from baseline volume in the placebo subjects (0%, p=0.0004).

A statistically significant improvement in Uterine Fibroid Symptom Severity Score (UFSQOL) was also achieved compared to placebo. The Uterine Fibroid Symptom Quality of Life Survey (UFSQOL) was utilized in this study. The UFSQOL assesses distress from both bleeding and the bulk symptoms of uterine fibroids. Bulk symptoms include distress associated with pelvic pressure, frequent urination and fatigue. Proellex®-treated subjects experienced a LOCF median 70.9% improvement while placebo-treated subjects reported a 37.5% improvement (p=0.0211).

1. A liquid or semi-solid non-aqueous oral pharmaceutical fill composition for capsule dosage form, said composition consisting of (a) a pharmaceutically effective amount of an unmicronized selective progesterone receptor modulator (SPRM) per capsule dosage unit (b) polyethylene glycol (PEG) and optionally (c) a preservative, preferably butylated hydroxytoluene (BHT).

2. The composition according to claim 1, wherein said capsule dosage form is a hard gelatin capsule.

3. The composition according to claim 1, wherein said capsule dosage form is a soft gelatin capsule.

4. The composition according to claim 1, wherein the SPRM is CDB-4124 and wherein CDB-4124 is preferably present in amount of about 12 mg per capsule.

5. The composition according to claim 1, wherein the PEG is PEG 1000.

6. A gelatin capsule suitable for oral administration containing a non-aqueous capsule fill composition consisting essentially of about 12 mg of unmicronized CDB-4124 in liquid or semi-solid solution with PEG 1000, wherein the fill composition optionally comprises BHT.

7. The gelatin capsule according to claim 6, wherein the gelatin capsule is a hard gelatin capsule.

8. The gelatin capsule according to claim 6, wherein the gelatin capsule is a soft gelatin capsule.

9. A method of treating a progesterone-dependent disorder comprising orally administering to a subject in need thereof a liquid oral pharmaceutical composition in capsule dosage form, said composition consisting of (a) a pharmaceutically effective amount of an unmicronized selective progesterone receptor modulator (SPRM) per capsule dosage unit (b) polyethylene glycol (PEG) and optionally (c) a preservative, preferably BHT.

10. The method of claim 9, wherein the progesterone-dependent condition is selected from the group consisting of endometriosis and pain associated therewith, adenomyosis, endometriomas of the ovary, dysmenorrhea, uterine fibroids, endometrial hyperproliferation, ovarian cancer, and cervical cancer.

11. The method of claim 9, wherein the composition is administered once per day.

12. The method of claim 9, wherein the progesterone-dependent condition to be treated is uterine fibroids and wherein the composition is administered once per day for about 18 weeks, after which administration of the composition is discontinued for an off drug interval of sufficient duration to allow menses, and thereafter the composition is administered once per day for about 18 weeks, optionally followed by one or more additional 18 week treatment cycles.

13. A method of making a solid oral dosage form of a pharmaceutical composition comprising an effective amount of an SPRM and a PEG, the method comprising adding a molten solution of SPRM and said PEG to hard gelatin capsules and allowing said molten solution to cool therein, with the proviso that the SPRM is not micronized.
